(19) United States
(12) Patent Application Publication
(10) Pub. No.: US 2004/0162492 A1
(43) Pub. Date: Aug. 19, 2004

(54) Diagnosis supporting device

(75) Inventor: Hiroyuki Kobayashi, Saitama-ken (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 10/759,209

(22) Filed: Jan. 20, 2004

(30) Foreign Application Priority Data
Feb. 18, 2003 (JP) P2003-039548

(51) Int. Cl. A61B 6/00; A61B 1/06

(52) U.S. Cl. 600/476; 600/160; 600/180; 600/182; 382/128

(57) ABSTRACT

Disclosed is a diagnosis supporting device that acquires a reference image signal of a subject that is illuminated with reference light and a fluorescent image signal of the subject that is excited by irradiation with excitation light, calculates a first intensity coefficient based on the maximum brightness level of the fluorescent image data and calculates a second intensity coefficient corresponding to the maximum brightness level of the reference image data, and controls the intensities of the excitation light and the reference light according to the first and second intensity coefficients. The coefficients are determined such that the intensities of the excitation light and the reference light increase as the maximum brightness levels of the fluorescent image data and the reference image data decrease.

### BACKGROUND OF THE INVENTION

The present invention relates to a diagnosis supporting device for generating an image signal of an image of a subject used in a diagnosis of subcutaneous living tissue under an inner wall (a body cavity wall) of an esophagus, a bronchial tube or the like.

Irradiation of light at a specific wavelength excites living tissue, which causes living tissue to emit fluorescence. Further, intensity of fluorescence emitted from abnormal living tissue that is suffering from a lesion such as a tumor or cancer is smaller than that emitted from normal living tissue. Such a phenomenon also occurs in subcutaneous living tissue under a body cavity wall.

U.S. Pat. No. 6,371,908 discloses a diagnosis supporting device that finds abnormality of subcutaneous living tissue under a body cavity wall through the use of the phenomenon. A diagnosis supporting device of such a type displays a special observation image on a monitor. The special observation image shows an affected area in a predetermined color (for example, red) on a monochromatic image of a body cavity.

The diagnosis supporting device alternatively emits visible light (reference light) within a predetermined narrow wavelength band to illuminate a body cavity and excitation light to excite living tissue through a fiber bundle led through an endoscope. The diagnosis supporting device specifies positions of pixels that should be displayed as affected areas by comparing fluorescent image data that is acquired by the endoscope during the irradiation of the excitation light and reference image data that is acquired by the endoscope during the illumination of the reference light. Then the diagnosis supporting device generates color image data based on the reference image data and converts the color of the specified pixels in the color image data into red, thereby image data of a special observation image is generated.

The diagnosis supporting device determines whether a pixel should be displayed as an affected area or not by comparing a brightness level of the pixel in the fluorescent image data and a brightness level of the pixel at the corresponding position in the reference image data. Namely, the diagnosis supporting device determines whether a pixel should be displayed as an affected area or not by comparing the intensity of the fluorescent light emitted from a position on the body cavity wall with the intensity of the reference light reflected from the same position on the body cavity wall. In the conventional diagnosis supporting device, the illumination area of the reference light on the body cavity wall is almost coincident with that of the excitation light so as not to cause errors in the comparisons.

While the intensity of the fluorescent light emitted from living tissue is extremely weak as compared with that of the excitation light irradiated to the living tissue, the intensity of the fluorescent light tends to be proportional to that of the excitation light. Therefore, it is necessary to irradiate the living tissue with the excitation light as strong as possible to sharpen an image based on the fluorescent image data acquired by the diagnosis supporting device.

U.S. Pat. No. 6,537,211 discloses a diagnosis supporting device that increases a voltage applied to a light source within a permissible range to increase the intensity of the excitation light only when the excitation light irradiates living tissue.

Incidentally, the intensity of the reference light reflected from a surface of a body cavity wall is extremely stronger than the intensity of the fluorescent light emitted from the body cavity wall. Therefore, it is necessary to control the intensity of the reference light in such a conventional diagnosis supporting device so as not to cause errors in the comparison of the fluorescent image data with the reference image data. A mechanical aperture may be used to control the intensity of the reference light.

However, the control by the mechanical aperture may cause inconsistency in the irradiation areas of the reference light and the excitation light. Such inconsistency causes errors in the comparison of the fluorescent image data with the reference image data, which causes a problem that the affected area determined by the comparison does not show the real affected area.

### SUMMARY OF THE INVENTION

It is therefore an object of the present invention to provide an improved diagnosis supporting device that is capable of controlling intensity of reference light without changing an irradiation areas of excitation light and reference light.

A diagnosis supporting device of the present invention is connected to an endoscope system that captures an image of a subject faced to the tip of an endoscope to generate special observation image data for displaying a special observation image for diagnosis based on various image data transmitted from the endoscope system.

The diagnosis supporting device of the present invention includes a light emitting section that alternately emits excitation light to excite living tissue and reference light to illuminate the subject, a probe that is inserted through a forceps channel to guide the excitation light and the reference light from a proximal end to a distal end, an image data acquiring section that acquires fluorescent image data generated by the endoscope system when the light emitting section emits the excitation light and reference image data generated by the endoscope system when the light emitting section emits the reference light, an intensity measuring section that extracts the maximum brightness level from the brightness levels of all the pixels in the fluorescent image data and extracts the maximum brightness level from the brightness levels of all the pixels in the reference image data whenever the image signal acquiring section acquires a set of the reference image data and the fluorescent image data, a calculating section that calculates a first intensity coefficient based on the maximum brightness level of the fluorescent image data according to a first operational expression and that calculates a second intensity coefficient corresponding to the maximum brightness level of the reference image data according to a second operational expression, and a light controller that controls the intensity of the excitation light according to the first intensity coefficient and that controls the intensity of the reference light according to the second intensity coefficient. The first and second operational expressions are determined such that the intensities of the excitation light and the reference light increase as the maximum brightness levels of the fluorescent image data and the reference image data decrease.

With this construction, the intensities of the excitation light and the reference light are controlled based on the maximum brightness levels in the fluorescent image data and the reference image data acquired by the image acquiring section. Therefore, when the relationship between the maximum brightness level in the fluorescent image data and the intensity of the excitation light, and the relationship between the maximum brightness level in the reference image data and the intensity of the reference light are predetermined, that is, when the first and second operational expressions are appropriately determined, the area shown as an affected area on the special observation image displayed on a monitor based on the special observation image data is coincident with a actual affected area.

The light emitting section may include a light source that varies intensity of the light in response to voltage applied to the light source. In such a case, the light controller controls the intensities of the excitation light and the reference light by changing the voltage applied to the light source.

The diagnosis supporting device of the present invention may further include an affected-area-information acquiring section that determines whether a difference between brightness level of a pixel in the reference image data and brightness level of a pixel in the fluorescent image data at the corresponding position is larger than a predetermined threshold value or not for all of the pixels in the reference image data whenever the image signal acquiring section acquires a set of the reference image data and the fluorescent image data, and that acquires position information that specifies the positions of the pixels whose differences are larger than the threshold value, an image generating section that generates color image data for displaying a monochromatic image on a monitor based on the reference image data acquired by the image data acquiring section, an image composing section that composes the color image data generated by the image generating section and the position information to convert the pixels on the color image data that are represented by the position information into specified pixels exhibiting a predetermined color, and an output section that output the composed color image data composed by the image composing section as special observation image data.

With this construction, an operator can specify an outline and unevenness of body cavity wall through the special observation image data and can specify parts that have high risk to be suffering from a lesion such as a tumor or cancer through maculate red parts and/or block parts of the predetermined color (red, for example) in the special observation image data.

### DESCRIPTION OF THE ACCOMPANYING DRAWINGS

FIG. 1 is a block diagram showing an endoscope system of an embodiment according to the present invention;

FIG. 2 shows details of a light emitting section of the diagnosis supporting device shown in FIG. 1;

FIG. 3 is a timing chart of the outputs of the excitation light and the reference light, and a driving signal;

FIG. 4 is a block diagram showing an image processing section of the diagnosis supporting device of the embodiment;

FIG. 5 is a flowchart to show a process executed by the special-observation-image creating circuit in the image processing section;

FIG. 6A shows a graph showing relationships between a first intensity coefficient and the maximum brightness level of the fluorescent image data; and

FIG. 6B shows a graph showing relationships between a second intensity coefficient and the maximum brightness level of the reference image data.

### DESCRIPTION OF THE EMBODIMENTS

An embodiment of the present invention will be described hereinafter with reference to the drawings.

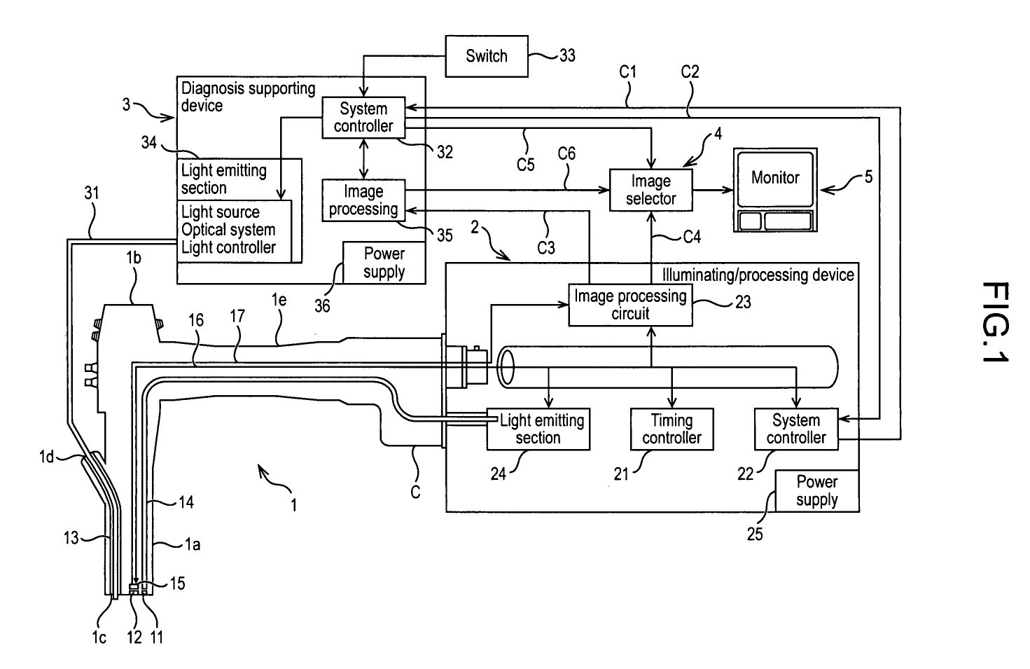

FIG. 1 is a block diagram of an endoscope system of the embodiment. The endoscope system is provided with a video endoscope 1, an illuminating/processing device 2, a diagnosis supporting device 3, an image selector 4 and a monitor 5.

At first, the video endoscope 1 will be explained. The video endoscope 1 has a flexible insertion tube 1a that can be inserted in a living body and an operating portion 1b on which angle knobs (not shown) to control a bending mechanism (not shown) built in the tip of the insertion tube 1a are mounted.

A distribution lens 11 and an objective lens 12 are built on the tip surface of the insertion tube 1a and a forceps opening 1c of a forceps channel 13 opens at the tip surface. The other forceps opening 1d of the forceps channel 13 opens at the side of the operating portion 1b. A treatment tool such as an electric scalpel may be inserted through the forceps channel 13.

An image of a subject formed through the objective lens 12 is taken by an image sensor 15. A light guide 14 for transmitting light to the distribution lens 11 and signal lines 16 and 17 connected to the image sensor 15 are led through the insertion tube 1a.

The light guide 14 and the signal lines 16 and 17 are also led through a flexible tube 1e that is extended from the insertion tube 1a at the side of the operating portion 1b, and proximal ends thereof are fixed to an end face of a connector C mounted on the proximal end of the flexible tube 1e.

Next, the illuminating/processing device 2 will be explained. The illuminating/processing device 2 includes a timing controller 21, a system controller 22, an image processing circuit 23, a light emitting section 24 and a power supply 25 supplying these circuits with electricity. Further, the illuminating/processing device 2 is provided with a connector-supporting portion (not show) to which the above-described connector C is fitted. Fitting the connector C to the connector-supporting portion, the proximal end of the light guide 14 is inserted into the light source 24, the signal line 16 is connected to the system controller 22 and the signal line 17 is connected to the image processing circuit 23.

The timing controller 21 generates various reference signals and controls the outputs of them. Various processes in the illuminating/processing device 2 are executed according to the reference signals.

The system controller 22 controls the entire system of the illuminating/processing device 2. The system controller 22 is connected to the diagnosis supporting device 3 through cables C1 and C2. The system controller 22 usually sends the reference signals to the diagnosis supporting device 3 through the cable C1. Further, the system controller 22 receives a changeover signal from the diagnosis supporting device 3 through the cable C2 and controls ON/OFF of the light emission of the light emitting section 24 in response to the changeover signal. Still further, the system controller 22 repeatedly sends out a driving signal to the image sensor 15 through the signal line 16 at a constant time interval defined by the reference signal while a main power supply keeps ON. Since the driving signal is usually transmitted without reference to the light emission of the light emitting section 24, the image sensor 15 repeatedly sends out the image data to the image processing circuit 23.

The image processing circuit 23 acquires the image signal transmitted from the image sensor 15 as an analog signal at an each timing represented by the reference signal. In the other words, the image processing circuit 23 continuously acquires the image data all the time. Three timings represented by the reference signals form one cycle. The image processing circuit 23 converts image data acquired at a first timing in one cycle into blue (B) component image data, converts image data acquired at a second timing in the cycle into red (R) component image data and converts image data acquired at a third timing in the cycle into green (G) component image data. Then the image processing circuit 23 outputs respective color component image data as three (R, G and B) analog color component signals to the diagnosis supporting device 3 through a cable C3. In addition, the image processing circuit 23 outputs an analog composite video signal such as a PAL signal or an NTSC signal to the image selector 4 through a cable C4.

The light emitting section 24 is designed for a so-called frame-sequential method. The light emitting section 24 is provided with a light source that emits white light, an RGB rotation wheel that has color filters for R, G and B components, a condenser lens and a shutter. The RGB rotation wheel rotates such that the respective filters are alternately inserted in the optical path of the white light. The blue light, red light and green light transmitted through the filters are condensed by the condenser lens to be sequentially incident on the proximal end of the light guide 14. The blue light, red light and green light are guided by the light guide 14 and are diffused by the distribution lens 11 to illuminate the subject faced to the tip of the video endoscope 1. Then, an image of the subject formed by blue light, an image of the subject formed by red light and an image of the subject formed by green light are sequentially formed on the image-taking surface of the image sensor 15.

The image sensor 15 converts the images of the subject formed by blue, red and green lights into the analog image data, which are referred to as blue image data, red image data and green image data, respectively. The converted analog image data is transmitted to the image processing circuit 23 through the signal line 17.

The light emitting section 24 is controlled by the system controller 22 to synchronize the timings at which the blue light, red light and green light are incident on the light guide 14 with the first, second and third timings represented by the reference signals. Therefore, the B-component image data is generated from the blue image data, the R-component image data is generated from the red image data and the G-component image data is generated from the green image data. The image processing circuit 23 converts the acquired color image data into an RGB video signal, and then, converting the RGB video signal into an NTSC video signal or a PAL video signal.

Next, the diagnosis supporting device 3 will be described. The diagnosis supporting device 3 is provided with a probe 31, a system controller 32, a switch 33, a light emitting section 34, an image processing circuit 35 and a power supply 36 supplying these circuits with electricity.

The probe 31 is multiple flexible optical fibers bundled with one another or a single flexible optical fiber through which ultraviolet light and visible light can transmit and a sheath covering the optical fiber(s). The probe 31 is led through the forceps channel 13 of the video endoscope 1 so that the tip end of the probe 31 is projected from the tip surface of the insertion portion 1a.

The system controller 32 controls the entire system of the diagnosis supporting device 3. The switch 33, which is an external foot switch or an operation switch mounted on a operation panel (not shown), is connected to the system controller 32. The system controller 32 changes a mode between a normal observation mode and a special observation mode in response to the condition of the switch 33. The system controller 32 is connected to the system controller 22 of the illuminating/processing device 2 through the cable C2, sending out a first changeover signal representing the normal observation mode or a second changeover signal representing the special observation mode to the system controller 22 of the illuminating/processing device 2. The system controller 22 controls the light emitting section 24 to emit light when the first changeover signal is input and to stop emission of light when the second changeover signal is input.

Further, the reference signal output from the system controller 22 of the illuminating/processing device 2 is usually input into the system controller 32 through the cable C1. The system controller 32 controls the light emitting section 34 and the image processing circuit 35 according to the reference signal in the special observation mode and stops these controls in the normal observation mode. Further, the system controller 32 is connected to the image selector 4, sending out the first and second changeover signals to the image selector 4.

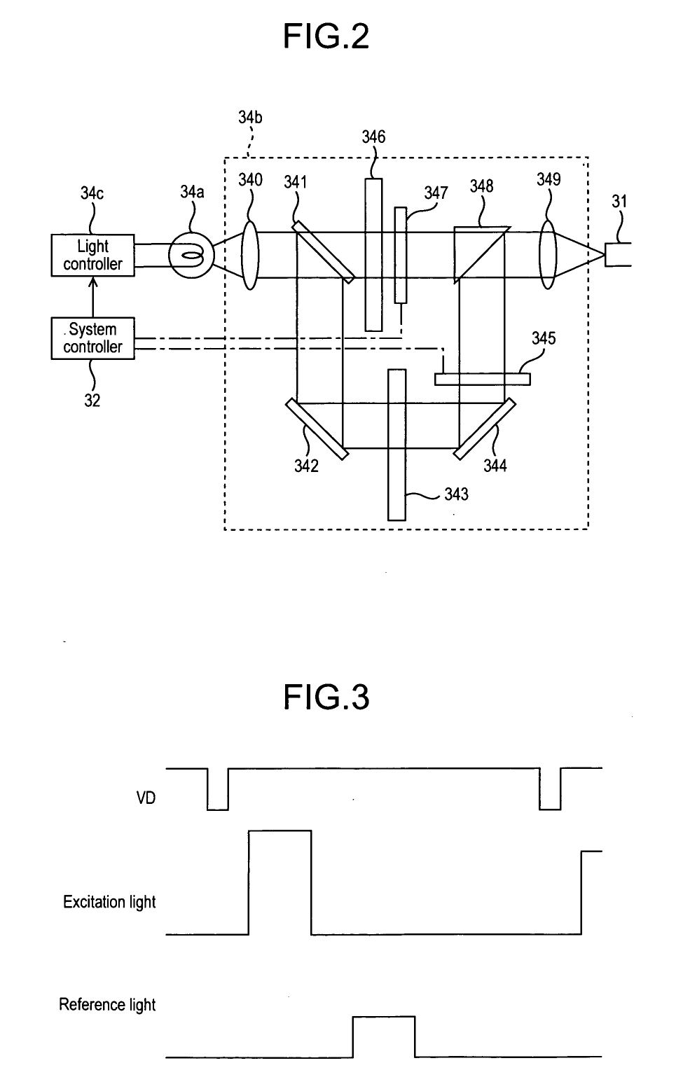

The light emitting section 34 makes ultraviolet light (the excitation light) to excite living tissue and the visible light within a predetermined narrow band (the reference light) be incident on the proximal end of the probe 31. FIG. 2 shows the details of the light emitting section 34. As shown in FIG. 2, the light emitting section 34 is provided with a light source 34a to emit light including the reference light and the excitation light, an optical system 34b to make the light emitted form the light source 34a be incident into the proximal end of the prove 31, and a light controller 34c to control intensity of the light emitted form the light source 34a.

The optical system 34b includes a collimator lens 340, a dichroic mirror 341, a first mirror 342, an excitation filter 343, a second mirror 344, an excitation-light shutter 345, a reference-light filter 346, a reference-light shutter 347, a beam combiner 348 and a condenser lens 349.

Divergent light emitted from the light source 34a is converted into a parallel beam through the collimator lens 340, being incident on the dichroic mirror 341. Light including the excitation light is reflected by the dichroic mirror 34 directed to the first mirror 342 and light including the reference light passes through the dichroic mirror 341. The light reflected by the dichroic mirror 341 is further reflected by the first mirror 342 and is incident on the excitation filter 343. The excitation light passed through the excitation filter 343 is reflected by the second mirror 344. When the excitation-light shutter 345 opens, the excitation light is reflected by the beam combiner 348, being converged by the condenser lens 349 to be incident on the proximal end of the probe 31. The light passed through the dichroic mirror 341 is incident on the reference-light filter 346. When the reference-light shutter 347 opens, the reference light passed through the reference-light filter 346 passes through the beam combiner 348, being converged by the condenser lens 349 to be incident on the proximal end of the probe 31.

Further, the open-close actuations of the excitation-light shutter 345 and the reference-light shutter 347 are controlled by the system controller 32 through respective actuators or drivers (not shown). Specifically, the excitation-light shutter 345 opens in response to the first timing of the reference signal and closes in response to the second and third timings. On the other hand, the reference-light shutter 347 opens in response to the second timing and closes in response to the first and third timings. Accordingly, the excitation light and the reference light are alternately incident on the proximal end of the probe 31.

The light controller 34c controls voltage of electricity supplied from the power supply 36 to the light source 34a. The light controller 34c is connected to the system controller 32, changing the voltage supplied to the light source 34a under the control of the system controller 32 to control the intensity of light emitted from the light source 34a. The system controller 32 instructs the light controller 34c to increase the intensity of the light emitted from the light source 34a from the minimum reference intensity to a predetermined intensity at the first and second timings. FIG. 3 is a timing chart that shows a relationship among the timing of incidence of the excitation light on the proximal end of the probe 31, the timing of incidence of the reference light on the proximal end of the probe 31 and the timing of the driving signal (VD) that shows one cycle. The vertical axis of FIG. 3 for the excitation light and the reference light indicates the intensity of the light being incident on the proximal end of the probe 31. As shown in FIG. 3, the excitation light is incident on the probe 31 at the first timing and the reference light is incident on the probe 31 at the second timing. At the other timing, since the shutters 345 and 347 are closed, the light intensity becomes zero. The intensity of the excitation light at the first timing and the intensity of the reference light at the second timing are determined by the system controller 32 based on intensity coefficients transmitted from the image processing circuit 35. Since the values of the intensity coefficients vary every cycle as described below, the intensities at the first and second timings determined by the system controller 32 vary every cycle. The light source 34a may emit light at the minimum reference intensity or may stop the emission of light at the timing other than the first and second timings. The latter is preferable to reduce power consumption.

As described above, since the light emitting section 34 makes the reference light and the excitation light be incident on the proximal end of the probe 31 by turn, a body cavity wall as a subject is alternately irradiated with the reference light and the excitation light guided through the probe 31 when the body cavity wall faces to the tip end of the probe 31. The excitation light excites subcutaneous living tissue under the body cavity wall so that the living tissue emits fluorescence. The reference light is reflected from the surface of the body cavity wall. When the body cavity wall is not irradiated with the excitation light or the reference light, the body cavity wall does not emit or reflect light. The image of the subject that emits fluorescence, the image of the subject that reflects the reference light and the image of the subject that does not emit or reflect light are taken by the image sensor 15 at the first, second and third timings, respectively. The taken images are converted to fluorescent image data, reference image data and dark image data. These image data are sequentially transmitted as analog signals to the image processing circuit 23 in the illuminating/processing device 2 through the signal line 17.

In the normal observation mode, since the system controller 22 in the illuminating/processing device 2 receives input of the first changeover signal, the light emitting section 24 sequentially emits blue (B) light, red (R) light and green (G) light. At this time, the light emitting section 34 of the diagnosis supporting device 3 does not emit light. Accordingly, the blue image data, the red image data and the green image data are sequentially transmitted to the image processing circuit 23 in the illuminating/processing device 2 in the normal observation mode, so that the image processing circuit 23 generates three (B, R and G) analog color component signals to show an color image and an analog composite video signal. The analog color component signals are transmitted to the image processing circuit 35 in the diagnosis supporting device 3 through the cable C3 and the analog composite video signal is transmitted to the image selector 4 through the cable C4. Furthermore, the image processing circuit 35 in the diagnosis supporting device 3 does not operate in the normal observation mode even if it receives the RGB analog color component signals.

On the other hand, the system controller 22 in the illuminating/processing device 2 receives input of the second changeover signal in the special observation mode, so that the light emitting section 24 does not emit light. At this time, the light emitting section 34 in the diagnosis supporting device 3 alternately emits the excitation light and the reference light. Accordingly, the fluorescent image data, the reference image data and the dark image data are entered into the image processing circuit 23 in the illuminating/processing device 2. Then, the image processing circuit 23 converts the fluorescent image data, the reference image data and the dark image data into the B-component image data, the R-component image data and the G-component image data, respectively. The image processing circuit 23 generates three (RGB) analog color component signals and an analog composite video signal based on a set of three component image data, transmitting the RGB analog image signals to the image processing circuit 35 in the diagnosis supporting device 3 through the cable C3 and transmitting the analog composite video signal to the image selector 4 through the cable C4.

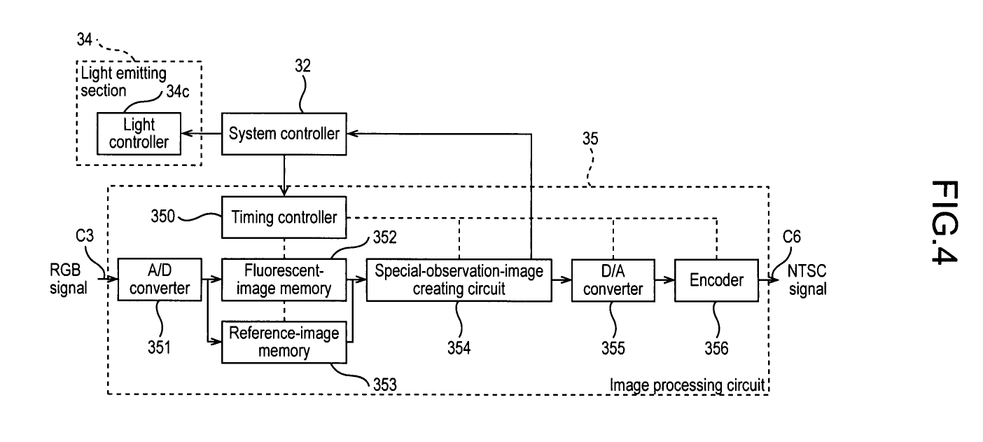

The image processing circuit 35 generates an image data that is used as a material of diagnosis (the special observation image data) through the use of the RGB analog color component signals transmitted from the image processing circuit 23 in the illuminating/processing device 2. FIG. 4 shows a general construction of the image processing circuit 35. As shown in FIG. 4, the image processing circuit 35 is provided with a timing controller 350, an analog/digital (A/D) converter 351, a fluorescent-image memory 352, a reference-image memory 353, a special-observation-image creating circuit 354, a digital/analog (D/A) converter 355 and an encoder 356. The A/D converter 351 and the memories 352, 353 correspond to the image data acquiring section.

The timing controller 350 receives the reference signal from the system controller 32, controlling the process in the image processing circuit 35 in response to the reference signal.

The A/D converter 351 is connected to the image processing circuit 23 in the illuminating/processing device 2 through the cable C3, converting the RGB analog color component signals fed from the image processing circuit 23 into digital color component signals.

Both the fluorescent-image memory 352 and the reference-image memory 353 are connected to the A/D converter 351. The fluorescent-image memory 352 stores the B-component of the RGB digital color component signals and the reference-image memory 353 stores the R-component thereof. Therefore, the fluorescent image signal and the reference image signal are stored in the fluorescent-image memory 352 and the reference-image memory 353, respectively. The special-observation-image creating circuit 354 reads the fluorescent image signal and the reference image signal from the memories 352 and 353 at a timing defined by the reference signal from the timing controller 350.

The special-observation-image creating circuit 354 has a ROM in which a program discussed below is stored, a CPU that executes the program read from the ROM, a RAM on which workspace of the CPU is developed or the like. The special-observation-image creating circuit 354 generates a special observation image data based on the fluorescent image data and the reference image data as described below, sending out the generated data as RGB digital color component signals to the D/A converter 355.

The D/A converter 355 converts the RGB digital color component signals fed from the special-observation-image creating circuit 354 into analog color component signals, respectively, sending out the converted signals to the encoder 356.

The encoder 356 converts the RGB analog color component signals fed from the D/A converter 355 into an analog composite video signal such as a PAL signal or an NTSC signal. Further, the encoder 356 is connected to the image selector 4 through the cable C6, sending out the analog composite video signal of the special observation image data to the image selector 4.

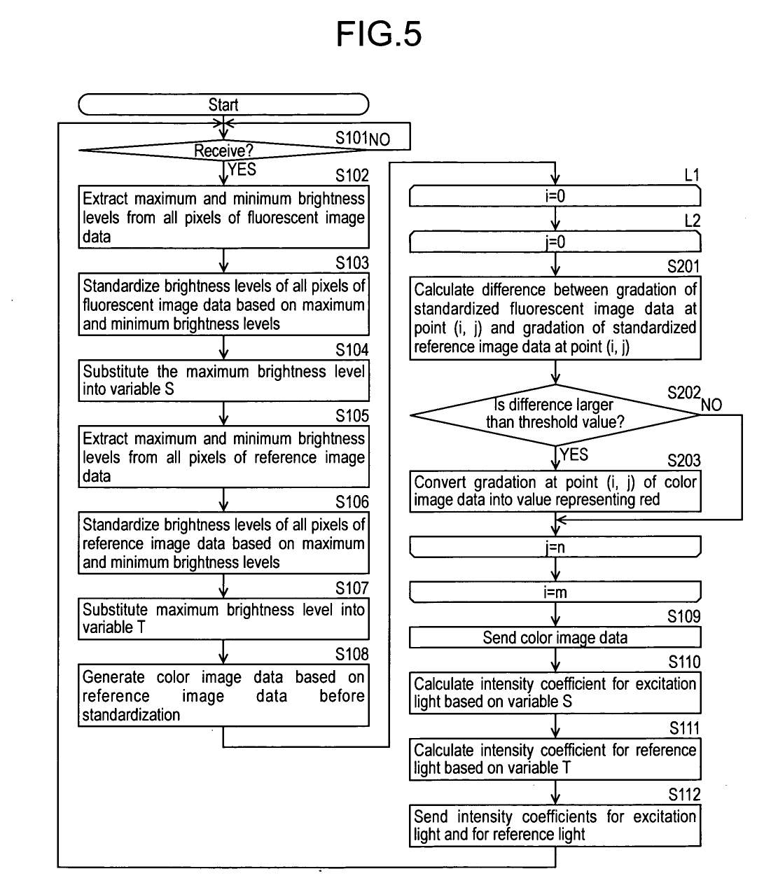

The process executed by the special-observation-image creating circuit 354 will be described. The CPU in the special-observation-image creating circuit 354 reads a program from the ROM to execute the process as long as the main power is turned on. FIG. 5 is a flowchart showing the process.

After starting the process, the CPU waits receiving of fluorescent image data and reference image data transmitted from the respective memories 352 and 353 (S101).

When the CPU receives both the image data, the CPU extracts the maximum and minimum brightness levels from all the pixels of the fluorescent image data (S102). Then the CPU standardizes brightness levels of all pixels in the fluorescent image data by converting the maximum brightness level into the maximum gradation (for example, “255”), the minimum brightness level into the minimum gradation (for example, “0”) and intermediate brightness levels into the respective corresponding gradations (S103). A gradation of a pixel is equivalent to a standardized brightness level. Further, the CPU substitutes the maximum brightness level extracted at S102 into a variable S (S104).

Next, the CPU extracts the maximum and the minimum brightness levels from all the pixels of the reference image data (S105) and standardizes the brightness levels of all pixels in the reference image data in the same manner as the process at S103 (S106). Further, the CPU substitutes the maximum brightness level extracted at S105 into a variable T (S107).

Then the CPU generates color image data to display a monochromatic image on the monitor 5 based on the reference image data before standardization (S108).

Assuming that points (i, j) on a two-dimensional coordinate system defined for all pixels of the fluorescent image data and the reference image data range from (0, 0) to (m, n), the CPU executes a first loop process L1 with incrementing “i” from “0” to “m” by “1”. In the first loop process L1, the CPU executes a second loop process L2 with incrementing “j” from “0” to “n” by “1”.

In the second loop process L2, the CPU calculates the difference of gradations at the point (i, j) by subtracting the gradation after standardization at the point (i, j) in the fluorescent image data from the gradation after standardization at the point (i, j) in the reference image data (S201). Then the CPU determines whether the difference at the point (i, j) is larger than a predetermined threshold value or not (S202). If the difference at the point (i, j) is equal to or larger than the predetermined threshold value (S202, YES), the CPU converts the gradation of the pixel at the point (i, j) in the color image data created at S108 into the gradation exhibiting predetermined color on the monitor (S203). For example, the RGB value of the converted pixel is (255, 0, 0) to exhibit red on the monitor. On the other hand, if the difference at the point (i, j) is smaller than the predetermined threshold value (S202, NO), the gradation of the pixel at the point (i, j) in the color image data created at S108 is retained.

After the CPU repeats the process from S201 to S203 for the points (i, 0) to (i, n), the process exits from the second loop process L2.

After the CPU repeats the second loop process L2 for the points (0, j) to (m, j), the process exits from the first loop process L1. Accordingly, the process from S201 to S203 is repeated for all points in the two-dimensional coordinate through the first and second loop processes L1 and L2.

After exiting from the first loop process L1, the CPU sends the color image data as the special observation image data to the D/A converter 355 (S109).

Then the CPU calculates a first intensity coefficient y₁ (S110) based on the value of the variable S that stores the maximum brightness level in the fluorescent image data according to the following first operational expression (1):

y₁=−α₁S+β₁ (1)

where α₁ and β₁ are predetermined constants. The first intensity coefficient y₁ is used for determining the intensity of light at the first timing (for taking a fluorescent image).

Next, the CPU calculates a second intensity coefficient y₂ (S111) based on the value of the variable T that stores the maximum brightness level in the reference image data according to the following second operational expression (2):

y₂=−α₂T+β₂ (2)

where α₂ and β₂ are predetermined constants. The second intensity coefficient y₂ is used for determining the intensity of light at the second timing (for taking a reference image).

After that, the CPU sends out the first and second intensity coefficients y₁ and y₂ calculated at S110 and S111 to the system controller 32 (S112). Then the CPU returns the process back to S101, waiting the inputs of the next fluorescent image data and the next reference image data fed from the memories 352 and 353.

According to the process of FIG. 5, the special-observation-image creating circuit 354 creates a special observation image data whenever it receives the inputs of the fluorescent image data and the reference image data from the fluorescent-image memory 352 and the reference-image memory 353, sending out the special-observation-image data to the D/A converter 355.

The special-observation-image creating circuit 354 is equivalent to the intensity measuring section when the circuit 354 executes the process at S102, S104, S105 and S107. Further, the special-observation-image creating circuit 354 is equivalent to the calculating section when the circuit 354 executes the process at S110 and S111. Still further, the special-observation-image creating circuit 354 that executes the process at S112, the system controller 32 and the light controller 34c are equivalent to the light controller.

The special-observation-image creating circuit 354 is equivalent to the affected-area-information acquiring section when the circuit 354 executes the process at S101 through S103, S105, S106, L1, L2 and S201. Further, the special-observation-image creating circuit 354 is equivalent to the image generating section when the circuit 354 executes the process at S108. Still further, the special-observation-image creating circuit 354 is equivalent to the image composing section when the circuit 354 executes the process at S202 and S203. Yet further, the special-observation-image creating circuit 354 is equivalent to the output section when the circuit 354 executes the process at S109.

Next, the function of the image selector 4 will be described. The image selector 4 receives the inputs of the first changeover signal corresponding to the normal observation mode, the second changeover signal corresponding to the special observation mode fed from the system controller 32 in the diagnosis supporting device 3.

The image selector 4 outputs the analog composite video signal fed from the image processing circuit 23 in the illuminating/processing device 2 to the monitor 5 to make the monitor 5 display the normal observation image in the normal observation mode. On the other hand, the image selector 4 outputs the analog composite video signal fed from the image processing circuit 35 in the diagnosis supporting device 3 to the monitor 5 to make the monitor 5 display the special observation image in the special observation mode.

Next, the operation of the above-described system according to the embodiment will be described. An operator turns on the main powers of the illuminating/processing device 2 and the diagnosis supporting device 3, operating the switch 33 to set the observation mode in the normal observation mode. Then the operator inserts the insertion portion 1a of the video endoscope 1 into body cavity of a subject, directing the distal end thereof to an area to be observed. The monitor 5 displays the color image of the area that is faced to the distal end of the video endoscope 1 as the normal observation image. The operator can know the condition of the body cavity wall while looking at the normal observation image.

Further, the operator observes the specific area, which is selected through the observation of the normal observation image, with the aid of the diagnosis supporting device 3. Specifically, the operator inserts the probe 31 of the diagnosis supporting device 3 into the forceps channel 13 from the forceps opening 1d so that the tip end of the prove 31 projects from the forceps opening 1c at the distal end of the video endoscope 1. Next, the operator operates the switch 33 to change the observation mode in the special observation mode. Then the excitation light and the reference light are alternately emitted from the tip end of the probe 31, and the image sensor 15 alternately takes the image of the subject that emits fluorescence and the image of the body cavity wall illuminated by the reference light. The special observation image data is repeatedly created based on the fluorescent image data and the reference image data acquired by the image taking, and the created special observation image data is sent to the monitor 5 as the analog composite video signal. The monitor 5 displays the monochromatic special observation image of the area that is faced to the distal end of the video endoscope 1. In the special observation image, the affected area is represented by a red area for example.

At the same time that the special observation image data is created, the first and second intensity coefficients y₁ and y₂, which are used to control the intensity of the excitation light and the reference light from the predetermined minimum reference intensity, are repeatedly calculated based on the fluorescent image data and the reference image data that are acquired by turns. The first and second intensity coefficients y₁ and y₂ are used to control the output of the light source 34a at the first and second timings, respectively. As a result, the intensities of the excitation light and the reference light that are incident on the proximal end of the probe 31 increase from the predetermined minimum reference intensity.

Since the increments of the light intensities of the excitation light and the reference light vary according to the values of the constants α₁, α₂, β₁ and β₂ defined in the expressions (1) and (2), when the values of these constants are determined so as not to cause errors in comparisons of the fluorescent image data and the reference image data, the actual affected area is properly shown as the affected area in the special observation image displayed on the monitor 5. Therefore, the operator can specify an outline and unevenness of body cavity wall while looking at the special observation image and can recognize living tissue that emits relatively weak fluorescence, i.e., the parts that have high risk to be suffering from a lesion such as a tumor or cancer, as maculate red parts and/or block red parts in the special observation image.

Since the first and second intensity coefficients y₁ and y₂ linearly decrease as the maximum brightness levels in the fluorescent image data and the reference image data increase as shown in the expressions (1) and (2), the rates of change of the first and second intensity coefficients y₁ and y₂ are identical to each other when the value of the constant α₁ is equal to the value of the constant α₂. However, since the intensity of the reference light reflected from the surface of the subject is larger than the intensity of the fluorescence emitted from the subject, the value of the constant β₁ must be larger than the value of the constant β₂.

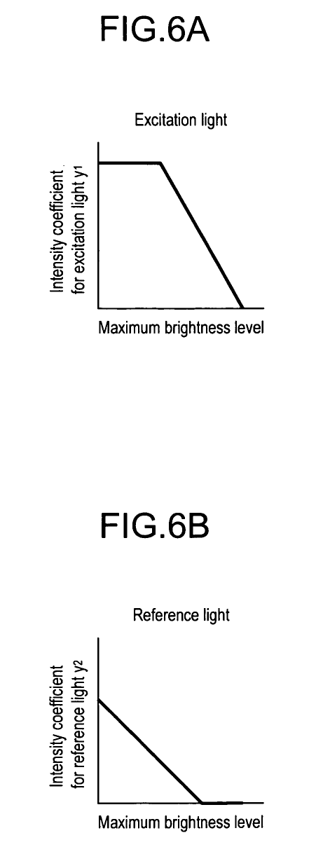

In the above-described embodiment, the first and second intensity coefficients y₁ and y₂ vary linearly in response to the maximum brightness levels. However, the coefficients may be determined according to the relationships shown in FIG. 6A and FIG. 6B.

As shown in FIG. 6A, the first intensity coefficient y₁ for the excitation light may be constant at the maximum value when the maximum brightness level in the fluorescent image data is smaller than the predetermined value. With this setting, the first intensity coefficient y₁ will become maximum when the brightness level of the fluorescent image data is too low, which can reduce possibility of the error in the comparison of the fluorescent image data and the reference image data. The maximum value of the intensity coefficient is determined to define the upper limit of the voltage applied to the light source 34a not to damage the light source 34a.

As shown in FIG. 6B, the second intensity coefficient y₂ for the reference light may be constant at the minimum value when the maximum brightness level in the reference image data is larger than the predetermined value. Since the intensity of the reference light reflected from the subject is larger than that of the fluorescence emitted form the subject, it is not always necessary that the intensity of the reference light increases. When the second intensity coefficient y₂ is set at the minimum value, the reference light is incident on the probe 31 at the minimum reference intensity at the second timing.

As described above, the present invention can provide an improved diagnosis supporting device that is capable of controlling the intensity of the reference light without changing an irradiation areas of excitation light and reference light.

The present disclosure relates to the subject matter contained in Japanese Patent Application No. P2003-039548, filed on Feb. 18, 2003, which are expressly incorporated herein by reference in its entirety.

What is claimed is:

1. A diagnosis supporting device connected to an endoscope system that captures an image of a subject faced to the tip of an endoscope to generate special observation image data for displaying a special observation image for diagnosis based on various image data transmitted from the endoscope system, said diagnosis supporting device comprising:
a light emitting section that alternately emits excitation light to excite living tissue and reference light to illuminate the subject;
a probe that is inserted through a forceps channel to guide the excitation light and the reference light from a proximal end to a distal end;
an image data acquiring section that acquires fluorescent image data generated by the endoscope system when the light emitting section emits the excitation light and acquires reference image data generated by the endoscope system when the light emitting section emits the reference light;
an intensity measuring section that extracts the maximum brightness level from the brightness levels of all the pixels in the fluorescent image data and extracts the maximum brightness level from the brightness levels of all the pixels in the reference image data whenever the image signal acquiring section acquires a set of the reference image data and the fluorescent image data;
a calculating section that calculates a first intensity coefficient based on the maximum brightness level of the fluorescent image data according to a first operational expression and that calculates a second intensity coefficient corresponding to the maximum brightness level of the reference image data according to a second operational expression; and
a light controller that controls the intensity of the excitation light according to the first intensity coefficient and that controls the intensity of the reference light according to the second intensity coefficient,
wherein said first and second operational expressions are determined such that the intensities of said excitation light and said reference light increase as the maximum brightness levels of said fluorescent image data and said reference image data decrease.

2. The diagnosis supporting device according to claim 2, wherein said light emitting section includes a light source that varies intensity of the light in response to voltage applied to said light source, and wherein said light controller controls the intensities of said excitation light and said reference light by changing the voltage applied to said light source.

3. The diagnosis supporting device according to claim 1, further comprising:
an affected-area-information acquiring section that determines whether a difference between brightness level of a pixel in said reference image data and brightness level of a pixel in said fluorescent image data at the corresponding position is larger than a predetermined threshold value or not for all of the pixels in said reference image data whenever said image signal acquiring section acquires a set of said reference image data and said fluorescent image data, and that acquires position information that specifies the positions of the pixels whose differences are larger than said threshold value;
an image generating section that generates color image data for displaying a monochromatic image on a monitor based on said reference image data acquired by said image data acquiring section;
an image composing section that composes said color image data generated by said image generating section and said position information to convert the pixels on said color image data that are represented by said position information into specified pixels exhibiting a predetermined color; and
an output section that output the composed color image data composed by said image composing section as special observation image data.

4. The diagnosis supporting device according to claim 3, wherein said specific pixels exhibit red.

5. The diagnosis supporting device according to claim 1, wherein said probe consists of a number of optical fibers that are bundled up with one another.
